(19) United States
(12) Patent Application Publication
(10) Pub. No.: US 2011/0212534 A1
(43) Pub. Date: Sep. 1, 2011

(54) METHODS FOR DETERMINING LEVELS OF 1,25 DIHYDROXY VITAMIN D2 AND D3

(76) Inventors: Robert L. Taylor, Rochester, MN (US); Ravinder J. Singh, Rochester, MN (US); Kendall W. Cradic, Byron, MN (US); James L. Bruton, JR., Henrizg, VA (US); Brian C. Netzel, Rochester, MN (US)

(21) Appl. No.: 13/058,695

(22) PCT Filed: Aug. 11, 2009
(86) PCT No.: PCT/US09/53399

§ 371 Date: May 16, 2011

Related U.S. Application Data
(60) Provisional application No. 61/087,793, filed on Aug. 11, 2008.

(51) Int. Cl. G01N 33/00; G01N 33/02; G01N 33/82

(52) U.S. Cl. 436/131

(57) ABSTRACT

Methods for determining the amount of DHVD2 and/or DHVD3 in a sample are provided. The methods can employ LC-MS/MS techniques coupled with sample affinity purification and derivatization steps. Methods for diagnosing vitamin D deficiencies are also provided.

### CLAIM OF PRIORITY

This application claims priority to U.S. Provisional Application Ser. No. 61/087,793, filed on Aug. 11, 2008, the entire contents of which are hereby incorporated by reference.

### TECHNICAL FIELD

This document relates to materials and methods for determining levels of 1,25 dihydroxyvitamin D2 (DHVD2) and 1,25 dihydroxyvitamin D3 (DHVD3) in a sample, and more particularly to materials and methods employing mass spectrometry (MS).

### BACKGROUND

Vitamin D is a generic designation for a group of fat-soluble structurally similar sterols. Vitamin D compounds are derived from dietary ergocalciferol (from plants, vitamin D2) or cholecalciferol (from animals, vitamin D3), or by conversion of 7-dihydrocholesterol to vitamin D3 in the skin upon UV-exposure. Vitamin D2 and D3 are subsequently 25-hydroxylated in the liver to form 25-hydroxyvitamin D2 (250HD2) and 25-hydroxyvitamin D3 (250HD3). 250HD2 and 250HD3 represent the main body reservoir and transport form of vitamin D. They are stored in adipose tissue or are tightly bound by a transport protein while in circulation, and are subsequently hydroxylated to the corresponding 1,25 dihydroxy forms in the kidney. 1,25 dihydroxyvitamin D2 (DHVD2) and 1,25 dihydroxyvitamin D3 (DHVD3) are potent calciotropic hormones involved in the regulation of both calcium and phosphate metabolism, and are inhibitors of parathyroid hormone (PTH).

1,25-dihydroxy vitamin D levels may be high in primary hyperparathyroidism and in physiologic hyperparathyroidism secondary to low calcium or vitamin D intake. Some patients with granulomatous diseases (e.g., sarcoidis) and malignancies contain nonregulated 1-alpha hydroxylase in the lesion may have elevated 1,25 dihydroxy vitamin D levels and hypercalcemia.

Accurate and sensitive measurement of DHVD2 and DHVD3 levels is useful to assess vitamin D status, but is difficult because of the analytes' lipophilicities, low circulating concentrations (picomolar), and structural similarities to each other and their 25-hydroxy precursors. Radioimmunoassay or colorimetric assays can have low specificity for each analyte, typically due to cross-specificity of the antibodies employed in the methods for both analytes.

### SUMMARY

This document provides materials and methods that can be used to measure the levels of DHVD2, DHVD3, or both (total DHVD) in a sample. For example, DHVD2 and DHVD3 can be selectively and sensitively detected and quantitated using methods employing affinity purification, analyte derivatization, and mass spectrometric (MS) techniques, as described herein. The inventors have found that the combination of the affinity purification and analyte derivatization steps eliminates sample interferences, provides increased sensitivities, and provides more accurate results than methods that employ only analyte derivatization. Thus, the described methods can facilitate reliable quantification of both DHVD2 and DHVD3 to 5 pg/mL or lower. The materials and methods are thus useful to aid in the diagnosis of vitamin D deficiencies or hypervitaminosis D, to monitor vitamin D replacement therapies, and to aid in the diagnosis of various disorders, e.g., hypercalcemia, chronic renal failure, hypoparathyroidism, sarcoidosis, granulomatous diseases, malignancies, primary hyperparathyroidism, and physiologic hyperparathryoidism.

In one embodiment, this document provides a method employing affinity purification of a sample comprising contacting the sample with at least one antibody to extract the DHVD2 and/or DHVD3 present in the sample, followed by derivatization of the extracted DHVD2 and/or DHVD3, e.g., with a triazolinedione such as 4-phenyl-1,2,4-triazoline-3,5-dione (PTAD), and detection of the DHVD2 and/or DHVD3 with MS. The method allows for the sensitive, accurate, and precise quantification of DHVD2, DHVD3, or both, in samples such as serum and plasma. The methods can optionally involve additional sample purification steps, e.g., protein precipitation or dissociation steps (e.g., with acetonitrile and/or acid) and/or delipidation steps (e.g., with dextran sulphate and magnesium chloride); centrifugation or chromatography (e.g., solid phase extraction, SPE) steps; and the use of deuterated internal standards, such as DHVD2-d6 and DHVD3-d3.

The at least one antibody can be an antibody that is specific for both DHVD2 and DHVD3, e.g., an antibody that is cross-reactive with both DHVD2 and DHVD3. In some embodiments, two antibodies can be used, wherein one antibody binds with higher specificity to DHVD2 and the other antibody binds with higher specificity to DHVD3. The antibody or antibodies can be bound to a solid support (e.g., a bead, well, or plate), or can be unbound. Unbound antibodies can be captured using secondary antibodies, e.g., bound to a solid support, or with other known capture methods.

Unlike immunoassay methods, the methods provided herein can be highly automated, can separately measure DHVD2 and DHVD3 if desired, and can use an internal standard to monitor recovery of the sample purification and derivatization processes. In addition, the methods can provide superior analytical performance as compared to immunoassays.

In general, one embodiment provides a method for determining an amount of DHVD2 in a sample. The method includes affinity purification of the DHVD2 with an antibody specific for the DHVD2, followed by derivatization of the DHVD2, e.g., with any Cookson-type reagent or triazolinedione compound, such as MBOTAD (4-[4-(6-methoxy-2-benzoxazolyl) phenyl]-1,2,4-triazoline-3,5-dione), DMEQTAD (4-[2-(6,7dimethoxy-4-methyl-3-oxo-3,4-dihydroquinoxalyl) ethyl]-1,2,4-triazoline-3,5-dione), MTAD (4-methyl-1,2,4-triazoline-3,5-dione), or PTAD, and detection with MS. The MS technique can employ atmospheric pressure chemical ionization (API) or electrospray ionization (ESI). The mass spectrometry technique can be a tandem mass spectrometry (MS/MS) technique, or a LC-MS/MS technique. The LC can include an on-line extraction of the sample. The LC-MS/MS technique can include the use of a triple quadrupole instrument in Multiple Reaction Monitoring (MRM), positive-ion mode, and can include a Q1 scan tuned to select a precursor ion that corresponds to the [M+H⁺] or [M°+H⁺] of DHVD2, wherein M° as used herein refers to the loss of water from a molecule.

In one embodiment, the amount of DHVD3 can be determined, separately or in addition to, the amount of DHVD2. The method includes affinity purification of the DHVD3 with an antibody specific for the DHVD3, followed by derivatization of the DHVD3, e.g., with a Cookson-type reagent or triazolinedione compound, as discussed above, such as PTAD, and detection. with MS. The MS technique can employ atmospheric pressure chemical ionization (API) or electrospray ionization (ESI). The mass spectrometry technique can be a tandem mass spectrometry (MS/MS) technique, or a LC-MS/MS technique. The LC can include an on-line extraction of the sample. The LC-MS/MS technique can include the use of a triple quadrupole instrument in Multiple Reaction Monitoring (MRM), positive-ion mode, and can include a Q1 scan tuned to select a precursor ion that corresponds to the [M°+H⁺] or [M+H⁺] of DHVD3.

In an embodiment where both DHVD2 and DHVD3 are detected, the affinity purification step can employ an antibody that is specific (e.g., cross-reactive) for both DHVD2 and DHVD3. Alternatively, two antibodies can be employed, one with specificity for DHVD2 and one with specificity for DHVD3. In such an embodiment, an LC-MS/MS technique can include a Q1 scan tuned to select, independently, precursor ions that correspond to the [M°+H⁺] or [M+H⁺] of DHVD2 and DHVD3. An LC-MS/MS technique can include monitoring MRM precursor-product ion pair transitions having m/z values of 586.3/314.2 for DHVD2 and 574.3/314.2 for DHVD3.

An appropriate internal standard, such as a deuterated DHVD2 or deuterated DHVD3, can be employed in any of the methods described herein. In one embodiment, DHVD3-d3 is employed. In another embodiment, DHVD2-d6 is employed. In some embodiments, both DHVD3-d3 and DHVD2-d6 are employed. The internal standard DHVD2-d6 has an MRM parent-daughter ion pair transition m/z value of 592.3/314.2; the internal standard DHVD3-d3 has an MRM parent-daughter ion pair transition m/z value of 577.3/317.2.

A sample can be a biological or non-biological sample. A sample can be a human biological sample, such as a blood, urine, lachrymal, plasma, serum, or saliva sample.

In another embodiment, a method for determining whether or not a mammal has a vitamin D deficiency is provided. The method includes determining the amount of DHVD2 and DHVD3 in a sample from the mammal. Any of the methods described herein can be used to determine these amounts.

In yet another embodiment, a method for determining whether or not a mammal has hypervitaminosis D is provided. The method includes determining the amount of DHVD2 and DHVD3 in a sample from the mammal.

Other features and advantages will be apparent from the following detailed description, and from the claims. Unless otherwise defined, all technical and scientific terms used herein have the meaning commonly understood by one of ordinary skill in the art to which this disclosure pertains. All publications, patent applications, patents, and other references mentioned herein are incorporated by reference in their entirety. In case of conflict, the present specification, including definitions, will control. The disclosed materials, methods, and examples are illustrative only and not intended to be limiting. Skilled artisans will appreciate that methods and materials similar or equivalent to those described herein can be used to practice the methods.

### DESCRIPTION OF DRAWINGS

FIG. 1 is a graph comparing the results of two MS methods for purifying and quantifying total DHVD (sum of DHVD2 and DHVD3) in a sample, one method employing solid phase extraction (SPE) and analyte derivatization prior to MS quantification, and a second method employing SPE, affinity purification and analyte derivatization of the total DHVD (Affinity Ext) prior to MS quantification. The results are plotted against results obtained using the DiaSorin™ radioimmunoassay (RIA) kit. This figure demonstrates that affinity purification can eliminate interferences.

FIG. 2 is a fragmentation pattern of 1,25D derivatized with PTAD.

FIG. 3 is a plot of the DHVD2 and DHVD3 measurements for 44,955 female patients plotted by age.

FIG. 4 is a plot of the DHVD2 and DVHD3 measurements for 18,884 male patients plotted by age.

FIG. 5 illustrates the total DHVD result frequency per month for over 65,000 patients, plotted by age.

### DETAILED DESCRIPTION

Materials and methods for determining the amount of DHVD2 and/or DHVD3 in a sample, such as a sample from a patient in a clinical setting, are provided. The methods can be highly automated to allow for the efficient analysis of a number of samples in minimal time. In addition, the methods can be highly sensitive and can allow for the accurate differentiation of DHVD2 and DHVD3, thus avoiding the under- or over-detection of one or both of the analytes by other methods. On-line and/or automated purification and extraction methods can be employed, further minimizing sample handling and optimizing run time.

A method described herein can include the use of mass spectrometry techniques, such as tandem mass spectrometry (MS/MS) techniques. In certain cases, a liquid chromatography tandem mass spectrometry (LS-MS/MS) technique can be used. A mass spectrometry technique can include the use of a triple quadrupole instrument in Multiple Reaction Monitoring, positive ion mode. Depending on the analyte of interest, a MS/MS technique can include a Q1 scan that is tuned to select precursor ions that correspond to the [M°+H⁺] or [M+H⁺] of DHVD2 and/or DHVD3. Precursor-product ion pairs transitions characteristic for DHVD2 and/or DHVD3 can be monitored. An internal standard, such as deuterated DHVD2 or deuterated DHVD3 (or both), can be added to any sample, e.g., to evaluate sample recovery, precision, and/or accuracy.

### Samples and Sample Preparation

A sample for analysis can be any sample, including biological and non-biological samples. For example, a sample can be a food (e.g., meat, dairy, or vegetative sample) or beverage sample (e.g., orange juice or milk). A sample can be a nutritional or dietary supplement sample. In certain cases, a sample can be a biological sample, such as a tissue (e.g., adipose, liver, kidney, heart, muscle, bone, or skin tissue) or biological fluid (e.g., blood, serum, plasma, urine, lachrymal fluid, or saliva) sample. The biological sample can be from a mammal. A mammal can be a human, dog, cat, primate, rodent, pig, sheep, cow, or horse.

A sample can be treated to remove components that could interfere with the mass spectrometry technique. A variety of techniques known to those having skill in the art can be used based on the sample type. Solid and/or tissue samples can be ground, purified, and extracted to free the analytes of interest from interfering components. In such cases, a sample can be centrifuged, filtered, and/or subjected to chromatographic techniques (e.g., solid phase extraction columns (SPE), C18 columns) to remove interfering components (e.g., cells or tissue fragments). In some cases, reagents known to precipitate, bind, or dissociate impurities or interfering components can be added. For example, whole blood samples can be treated using conventional clotting techniques to remove red and white blood cells and platelets. A sample can be de-proteinized. For example, a plasma sample can have serum proteins precipitated using conventional reagents such as acetonitrile, KOH, NaOH, or others known to those having ordinary skill in the art, optionally followed by centrifugation of the sample. A sample can be acidified to, e.g., dissociate DHVD binding proteins.

Samples can be subjected to an affinity purification step to purify DHVD2 and/or DHVD3. An affinity purification step can employ the addition to the sample of an antibody that is specific for DHVD2, DHVD3, or both, depending on the analyte(s) that are to be detected. For example, an antibody that is specific for both DHVD2 and DHVD3 (e.g., cross-reactive with DHVD2 and DHVD3) can be used if the total amount of both DHVD2 and DHVD3 (total DHVD) is to be determined. For example, mouse anti-1,25DHVD beads from IDS, catalog #AA-54061G7, which are cross-reactive with both DHVD2 and DHVD3, can be employed. An antibody specific for only one or the other analyte can also be used if only one analyte is to be determined. The antibody can be bound to a solid support (e.g., such as a bead, well, or plate, as known to those having ordinary skill in the art) or can be in solution. Antibodies in solution can be captured using methods known to those having ordinary skill in the art, e.g., secondary antibodies bound to a solid support. DHVD2 and DHVD3 analytes can be eluted from the antibodies using conditions known to those having ordinary skill in the art, e.g., the use of high salt solutions, pH changes, or alcoholic solutions (e.g., ethanol), etc.

The affinity-purified DHVD2 and/or DHVD3 can be derivatized prior to MS analysis. Derivatization can provide suitable sites for protonation or cationization of the DHVD analytes. Derivatization of DHVD2 and/or DHVD3 can be performed with a Cookson-type reagent or a dienophile, such as a triazolinedione, which can react with the DHVD2 and DHVD3 to selectively derivatize the molecules. Triazolinedione reagents are known to those having ordinary skill in the art. One example is 4-phenyl-1,2,4-triazoline-3,5-dione (PTAD). Others include MBOTAD, DMEQTAD, and MTAD, as discussed previously.

In certain cases, an internal standard can be added to a sample prior to sample preparation. Internal standards can be useful to monitor extraction/purification efficiency. For example, DHVD2 and DHVD3 can bind to serum proteins such as vitamin D-binding globulin. An internal standard can be added to a sample and allowed to equilibrate for a period of time, e.g., 5, 10, 15, 20, 25, 30, 60, 120 or more minutes. Equilibration temperature can be from about 10° C. to about 45° C., or any value in between (e.g., 15, 25, 30, 35, 37, 42, or 44° C.). In certain cases, equilibration can be at room temperature for about 15 minutes.

An internal standard can be any compound that would be expected to behave under the sample preparation conditions in a manner similar to that of one or more of the analytes of interest. For example, a stable-isotope-labeled version of an analyte of interest can be used, such as a deuterated version of an analyte of interest. While not being bound by any theory, the physicochemical behavior of such stable-isotope-labeled compounds with respect to sample preparation and signal generation would be expected to be identical to that of the unlabeled analyte, but clearly differentiable by mass on the mass spectrometer. In certain methods, deuterated DHVD2 (e.g., DHVD2-d6) and/or deuterated DHVD3 (e.g., DHVD3-d3) can be employed.

To improve run time and minimize hands-on sample preparation, on-line extraction and/or analytical chromatography of a sample can be used. On-line extraction and/or analytical chromatography can be used, e.g., in LC-MS/MS techniques. For example, in certain methods, a sample, such as a deproteinized plasma sample, can be extracted using an extraction column, followed by elution onto an analytical chromatography column. The columns can be useful to remove interfering components as well as reagents used in earlier sample preparation steps (e.g., to remove reagents such as acetonitrile). Systems can be co-ordinated to allow the extraction column to be running while an analytical column is being flushed and/or equilibrated with solvent mobile phase, and vice-versa, thus improving efficiency and run-time. A variety of extraction and analytical columns with appropriate solvent mobile phases and gradients can be chosen by those having ordinary skill in the art.

### Mass Spectrometry

After sample preparation, a sample can be subjected to a mass spectrometry (MS) technique. A mass spectrometry technique can use atmospheric pressure chemical ionization (APCI) in the positive ion mode or electrospray ionization (ESI) to generate precursor positive ions. Analytes of interest can exist as charged species, such as protonated molecular ions [M°+H^{+] or [M+H}⁺] in the mobile phase. During the ionization phase, the molecular ions are desorbed into the gas phase at atmospheric pressure and then focused into the mass spectrometer for analysis and detection. Additional information relating to atmospheric pressure chemical ionization is known to those of skill in the art; see U.S. Pat. No. 6,692,971.

MS analysis can be conducted with a single mass analyzer (MS) or a “tandem in space” analyzer such as a triple quadrupole tandem mass spectrometer (MS/MS). Using MS/MS, the first mass filter (Quadrople 1, Q1) can select, or can be tuned to select, independently, one or more of the molecular ions of DHVD2, DHVD3, and the internal standard. The second mass filter (Q3) is tuned to select specific product or fragment ions related to the analyte of interest. Between these two mass filtration steps, the precursor molecular ions can undergo collisionally-induced dissociation (CID) at Q2 to produce product or fragment ions. The previously-described mass spectrometry technique can also be referred to as multiple reaction monitoring, or MRM. In multiple reaction monitoring, both quadrupoles Q1 and Q3 can be fixed (or tuned) each at a single mass, whereas Q2 can serve as a collision cell.

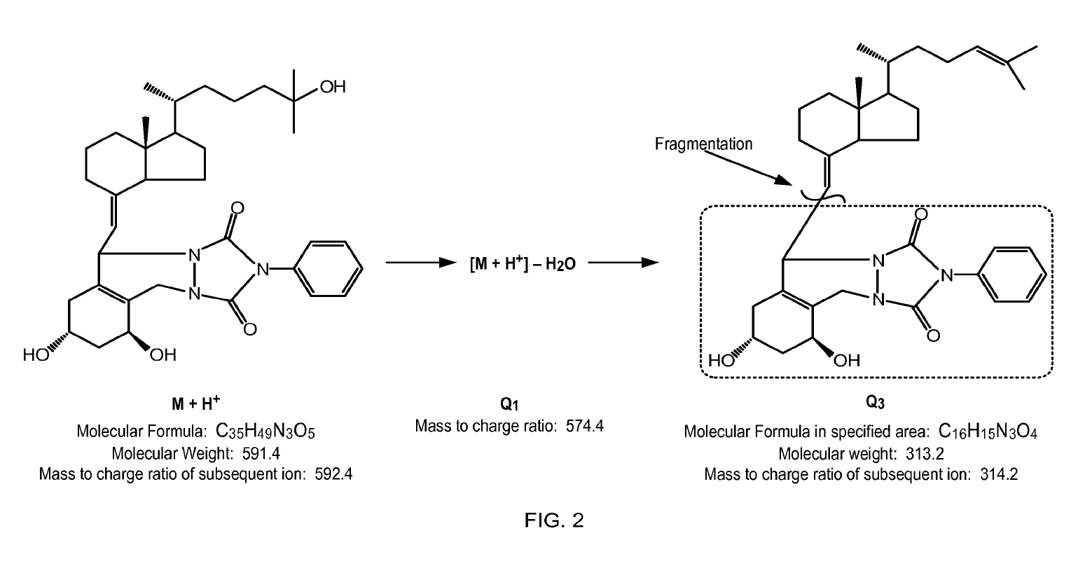

The precursor [M+H⁺] or [M°+H⁺] ions of DHVD2 and DHVD3 typically produce product ions that are shown in FIG. 2. Accordingly, precursor-product ion-pair transition can be 586.3/314.2 for DHVD2 and 574.3/314.2 for DHVD3.

An appropriate internal standard, such as a deuterated DHVD2 or deuterated DHVD3, can be employed in any of the methods described herein. In one embodiment, DHVD3-d3 is employed. In another embodiment, DHVD2-d6 is employed. In some embodiments, both DHVD3-d3 and DHVD2-d6 are employed. The internal standard DHVD2-d6 has an MRM parent-daughter ion pair transition m/z value of 592.3/314.2; the internal standard DHVD3-d3 has an MRM parent-daughter ion pair transition m/z value of 577.3/317.2.

The amount of each can be determined by comparing the area of precursor or product transitions, or both, of DHVD2 and/or DHVD3, with those of a standard calibration curve, e.g., a standard calibration curve generated from a series of defined concentrations of pure DHVD2 and/or DHVD3 standards. Variables due to the extraction and the LC-MS/MS instrumentation can be normalized by normalizing peak areas of the analyte of interest to the peak areas of the internal standard.

Any tandem MS machine and LC-MS/MS machine can be used, including the API 4000 triple quadrupole tandem mass spectrometer (ABI-SCIEX, Toronto, Canada). Software for tuning, selecting, and optimizing ion pairs is also available, e.g., Analyst Software Ver. 1.4 (ABI-SCIEX).

### Methods for Diagnosis

The methods described herein can be used in various diagnostic applications to monitor vitamin D-related pathologies, vitamin D and calcium homeostasis, and vitamin D replacement therapies. For example, the total amount of DHVD in a sample, such as a human patient sample, can be compared with clinical reference values to diagnose a vitamin D deficiency or hypervitaminosis D.

In one embodiment, a method for determining whether or not a mammal has a vitamin D deficiency is provided. The method can involve determining the amount of DHVD2 and DHVD3 in a sample from the mammal, such as a human. The amounts can be determined using any of the methods provided herein. In another embodiment, a method for determining whether or not a mammal has hypervitaminosis D is provided.

The method can involve determining the amount of DHVD2 and DHVD3 in a sample from the mammal using any of the methods described herein.

### EXAMPLES

### Example 1

### Representative Method for Determining DHVD2 and DHVD3 Levels in Serum or Plasmas

Deuterated stable isotopes (d₃-1,25 dihydroxyvitamin D3 and d₆-1,25 dihydroxyvitamin D2) are added to a 1.0-mL plasma sample as internal standards. 1,25-Dihydroxyvitamin D2 (DHVD2), 1,25-Dihydroxyvitamin D3 (DHVD3), and the internal standards are extracted from the sample using acetonitrile precipitation or acid dissociation of DHVD binding protein. The extracts are then further purified by SPE and affinity extraction. Extracts are then derivatized using 4-Phenyl-1,2,4-triazoline-3,5-dione (PTAD) and analyzed by LC-MS/MS using multiple reaction monitoring with a C18 turbo clean up for excess derivatizing reagent. DHVD2 and DHVD3 are quantified and reported individually and as a total DHVD, optionally with a clinical reference range attached to the total DHVD.

### A. REAGENTS/SUPPLIES

### 1. Standards

1α,25-Dihydroxyvitamin D2. Fluka Chemical Co. cat# 17944. 1 mg Store at −80 ° C.

Stable 15 years

1α,25-Dihydroxyvitamin D₃. Sigma Chemical Co. D1530. 0.1 mg. Store at −80 ° C.

Stable 15 years.

Stock Standard

200 ng/mL DHVD2 and 200 ng/mL DHVD3.

Store at −80 ° C. Stable 120 months.

### 2. Internal Standards

26, 26, 27, 27, 27-hexadeuterio-1α25-Dihydroxyvitamin D2. (DHVD2-d₆): Medical Isotopes catalog #D471. Store at −80 ° C. Stable 15 years

1α25-Dihydroxyvitamin-D3-[²H₃]. (DHVD3-d₃). Isosciences No catalog #, done as custom synthesis (Lot Number SL4-2006-051A1). Store at −80° C. Stable 15 years.

Internal Standard Stock (1 μg/mL DHVD3-d₃, 4 μg/mL DHVD2-d₆).

Dissolve 1 mg DHVD2-d₆and 0.25 mg DHVD3-d₃ in 200 proof absolute ethanol to a volume of 250 mL. Store at −80° C. in 10 mL aliquots in crimp top vials. Stable 120 months.

Internal Standard Working (2 ng/mL DHVD3-d₃, 8 ng/mL DHVD2-d₆).

Add 4 mL Internal Stock Standard to mL Reconstitution Solvent and dilute to a volume of 2 liters. Store at −20° C. Stable 120 months.

### 3. Solvents

Acetonitrile Burdick and Jackson (or other HPLC grade vendor) (4L) (FLAM #215908). Store ambient. Stable 1 year.

Isopropanol (2-propanol), HPLC Grade, JT Baker (FLAM #163212) (or other HPLC Grade vendor) Store ambient. Stable 1 year.

Ethanol (Ethyl Alcohol), 200 proof absolute, Aaper Alcohol and Chemical Company. Store ambient. Stable 1 year.

Acetone, HPLC Grade, Fisher Scientific (or other HPLC quality vendor) (FLAM #241994). Store ambient. Stable 1 year.

Hexane (HPLC grade) JT Baker HPLC JT9011-03 (FLAM #163211). Store ambient. Stable 1 year.

Methylene chloride (HPLC grade) (FLAM #229361). Store ambient. Stable 1 year. Methanol for extraction—Fisher Optima A-454-4 (FLAM #141352)

Methanol for Mass spec (GC Resolve), Fisher A-457-4 (4L) FLAM .

Store ambient. Stable 1 year.

CLRW (Clinical Laboratory Reagent Water), produced in-house using the NANOpure water system.

0.025% Acetic Acid. Add 5004, acetic acid to CLRW and bring to 2 liter volume.

Store ambient. Stable 1 week.

90:10 hexane: methylene chloride

3600 mL hexane and 400 mL methylene chloride. Made by Preparation and Processing. Store ambient. Stable 1 year.

70% Methanol. Made by Preparation and Processing. Store ambient. Stable 1 year.

Elution solvent: 90/10 hexane/isopropanol. Combine 4500 mL hexane and 500 mL isopropanol. Mix. Store ambient. Stable 1 year.

Reconstitution Solvent. 70/30 v/v Methanol/CLRW 1 μg/mL estriol. Add 140 mL of methanol and 200 μL 1 mg/mL estriol to a 200-mL volumetric flask. Fill to volume with CLRW. Store ambient. Stable 6 months.

### Mobile Phase:

Eluting Lines A: 0.025% acetic acid in CLRW.

Eluting Lines B: Methanol

Loading Lines A: 0.025% acetic acid in CLRW

Loading Lines B: Methanol

Loading Lines C: Cohesive Cleaning Solvent

Cohesive Cleaning Solvent: 45/45/10 (v/v/v) Acetonitrile/Isopropanol/Acetone.

Made by Preparation and Processing. Store ambient. Stable 1 year.

Cohesive Injector rinse solution 1: 98/2 (v/v) CLRW/Acetonitrile.

Made by Preparation and Processing.

Store ambient. Stable 1 year.

Cohesive Injector rinse solution 2: Cohesive Cleaning Solvent.

Made by Preparation and Processing.

Store ambient. Stable 1 year.

### 4. Chemicals

Estriol. Sigma catalog# E1253. Store ambient. Stable 10 years.

1 mg/mL estriol. Dissolve 25 mg estriol in methanol and bring to volume 25 mL.

Store −20° C. Stable 10 years.

PTAD (4-Phenyl−1,2,4-triazoline-3,5-dione). Sigma catalog#280992−1G. Store refrigerated. Stable 1 year.

Working PTAD solution. 20 μg/mL Dissolve 1 mg PTAD in 50 mLs acetonitrile.

Store ambient. Stable 12 hours.

Acetic Acid, EM Science, catalog#AX0073-75. Stable 2 years.

Hydrochloric acid (HCL), EM Science, catalog#HX0603-75. Stable 2 years.

0.2M HCL. Add 83.33 mL HCL to CLRW (at least 2 liters) and bring to volume of 5 liters with CLRW.

### 5. Miscellaneous

BSA (Albumin, bovine serum). Sigma A7888-50G. Store at 4° C. Stable 1 year.
1% BSA solution

Mouse anti−1,25 dihydroxyvitamin D Beads. IDS catalog#AA-54061G7 (for 1× solution). Store 4° C.
Assay wash buffer

| | | |
|---|---|---|
| 0.01M PO₄ | 1.10 | g NaH₂PO₄•H₂O (monobasic) |
| | 19.30 | g Na₂HPO₄•7H₂O (dibasic) |
| 0.5M NaCl | 232 | g NaCl |
| 0.1% Tween 20 | 8 | mL Tween 20 |

Dissolve in 7000 mL CLRW. Add Tween 20; pH to 7.4. Dilute up to 8 liters with CLRW.

### 6. Supplies

Cohesive C18 Extraction Column. 50×0.5 mm; Cohesive Technologies, Part #CH952817 (system number 192824-ParEx)

Phenomenex MAX-RP analytical column. 50 mm×2.0 mm, 4 μm, part #00B-4337-B0.

### Equipment

PE Sciex API 5000 LC-MS/MS with ElectroSpray Ionization Source Analyst Software 1.4

Cohesive TX4 on-line sample Preparation system.

System-96 Processor, positive pressure manifold. Chrom Tech, catalog#288-0001.

SPEWare 48 Place sample concentrator ChromTech catalog #279-0050

SPEWare 48 place Pressure Processor II Chrom Tech catalog #289-0004

96 Place sealing gasket for positive pressure manifold. Chrom Tech., catalog#278-0035.

### Calibration:

### Preparation Of Stock 1 Standard.

Dissolve the contents of 1 mg vial of DHVD2 in ethanol and quantitatively transfer to a 25-mL volumetric flask. Fill to volume with ethanol.

Dissolve the contents of 0.1mg vial of DHVD3 in ethanol and quantitatively transfer to a separate 10-mL volumetric flask. Fill to volume with ethanol.

Make 1:2, and 1:4 dilutions for DHVD2 (solution in step 1) and for DHVD3 (solution in step 2) so that there is at least 1 mL of each dilution.

Read the straight and each dilution on a UV spectrophtometer at 264 nm against an ethanol blank. The extinction coefficient for each is 18300, which calculates to an absorbency of 0.0183 for 1 μmole/L.

Calculate the concentration of each DHVD2 solution as follows: Abs.÷0.0183 μmole/L×412 μg/μmole×0.001 L/mL×dilution factor=μg/mL conc of DHVD2 stock

Calculate the concentration of each DHVD3 solution as follows: Abs.÷0.0183 μmole/L×400 μg/μmole×0.001 L/mL×dilution factor=μg/mL conc of DHVD3 stock.

Use the average concentration of the 3 solutions to assign a concentration to the DHVD2 stock and the DHVD3 stock.

Add 50 μg of DHVD2 and 50 μg of DHVD3 to a 250-mL volumetric flask and fill to volume with Reconstitution Solvent.

### B. Procedure:

### Acid Dissociation of DHVD Binding Protein.

Note: In some embodiments, the acetonitrile crash described below may be use to replace Acid Dissociation of DHVD Binding Protein

Label three 13×100 mm glass tube for each sample, standard, and control. Prepare any samples that need diluting using 1% BSA.

Pipet 1 mL of each sample, standard and control into the 1^{st} set of appropriately labeled 13×100 glass tubes.

Add 100 μL of working internal standard to each tube. Vortex on a multi-tube vortexer for 10 seconds at a setting of 5.

Incubate 15 minutes at room temperature.

Add 1.0 mL 0.2N HCL to each standard, control and sample.

Vortex on a multi-tube vortexer for 10 seconds at a setting of 5.

### Acetonitrile Crash

Label three 13×100 mm glass tube for each sample, standard, and control. Prepare any samples that need diluting using 1% BSA.

Pipet 1 mL of each sample, standard and control into the 1^{st} set of appropriately labeled 13×100 glass tubes.

Add 100 μL of working internal standard to each tube. Vortex on a multi-tube vortexer for 10 seconds at a setting of 5.

Incubate 15 minutes at room temperature.

Add 1.0 mL of acetonitrile and vortex for 30 seconds on a multi-tube vortexer at a setting of 6.

Centrifuge for 10 minutes at 2000 rpm in a large floor model centrifuge.

Pour supernatant of centrifuged extracts into 2^{nd} set of appropriately labeled 13×100 glass tubes.

Add 1.0 mL CLRW to each tube

### SPE

Use positive pressure manifold to perform SPE.
Condition cartridges with 2 mL methanol.
Apply acidified sample (or supernatant of ACN Crash) to C 18/OH SPE cartridges.

Wash cartridges with 5 mL 70% methanol. Wash cartridges with 2 mL 90:10 hexane:methylene chloride

Elute cartridges with 5 mL 90/10 hexane/isopropanol and collect in 13×100 glass tubes.

Dry extracts on Turbo Vap at 45° C. with nitrogen.

Affinity purification

Add 250 μL mouse anti-1,25 dihydroxyvitamin D Beads to each extracted sample, standard and control.

Incubate 45-90 minutes at room temperature on an orbital shaker at 230 rpm.

Label wells of fitted filter plate to match extracts.

Quantitatively transfer the mouse anti−1,25 dihydroxyvitamin D Beads of each sample, standard and control to appropriate well of fitted filter plate, using a pipette.

Use positive pressure manifold to remove liquid from wells of filter plate set at max flow.

Add 500 μL assay wash buffer to each well and remove liquid from wells of filter plate using positive pressure manifold.

Add 750 μL CLRW to each well and remove liquid from wells of filter plate using positive pressure manifold. Repeat.

Remove filter plate from positive pressure manifold and tap to make sure all water on the sides of the wells has gone into the gel at the bottom.

Place filter plate on positive pressure manifold and allow nitrogen to flow through for at least 2 minutes to dry the gel.

Remove filter plate and waste container from positive pressure manifold. Dry the bottom of the filter plate by tapping it on a paper towel.

Label 96 deep well plate to match fitted filter plate.

Place 96 deep well collection plate under filter plate.

Add 250 μL it ethanol to each well of filter plate and allow to stand for 2 minutes and drip into deep well collection plate.

Add 100 μL ethanol to each well of filter plate.

Use positive pressure manifold at a setting of 5 psi to push ethanol through the filter plate into the 96 deep well collection plate.

Place plate on plate dryer and evaporate until dryness with nitrogen. Set temperature to 75° C. and gas flow rate to 60. Note it is important to completely evaporate to dryness as ethanol can interfere with the following derivatization.

Prepare preview by pipetting 500 μL of working internal standard into 2 13×100 glass tubes and dry on a Turbo Vap. Previews are only run once per day.

### Derivatization

Add 250 μL of working PTAD derivatizing reagent to each well of collection plate. If it is the first plate of the day, also add 250 μL working PTAD to a blank well in the collection plate and to preview tubes. Transfer solution in preview tubes to deep well collection plate and place on an orbital shaker for 15 minutes at 150 rpm at room temperature.

Place plate on plate dryer and evaporate to dryness with nitrogen. Set temperature to 75° C. and gas flow rate to 60.

Reconstitute extracts in collection plate with 75 μL Reconstitution Solvent and place on orbital shaker for 15 minutes at 150 rpm. Preview and BLK wells are reconstituted with 375 μL reconstitution solvent.

Initiate analysis using the using the following parameters.

### MS Parameters:

MS Settings

| Polarity | Positive |
|---|---|
| ION Source | ElectroSpray |
| Resolution Q1 | Unit |
| Resolution Q3 | Unit |
| MR pause | 5 msec |
| DHVD3 Q1 Mass | 574.3 |
| DHVD3 Q3 Mass | 314.2 |
| Dwell Time | 150 ms |
| DHVD2 Q1 Mass | 586.3 |
| DHVD2 Q3 Mass | 314.2 |
| Dwell Time | 150 |
| DHVD3-d3 Q1 Mass | 577.3 |
| DHVD3-d3 Q3 Mass | 317.2 |
| Dwell Time | 150 |
| DHVD2-d6 Q1 Mass | 592.3 |
| DHVD2-d6 Q3 Mass | 314.2 |
| Dwell Time | 150 |
| Curtain Gas | 40 |
| GS1 | 60 |
| GS2 | 55 |
| Temperature | 650 |
| ihe | on |
| CAD | 7 |
| IS | 5500 |
| DP | 120 |
| EP | 10 |
| CE | 20 |
| CXP | 15 |

### Cohesive Solvent Lines:

Load 1A, Load 2A, Load 3A, Load 4A: Methanol

Elute 1A, Elute 2A, Elute 3A, Elute 4A: Methanol

Load 1 B, Load 2B, Load 3B, Load 4B: 0.025% Acetic Acid

Elute 1B, Elute 2B, Elute 3B, Elute 4B: 0.025% Acetic Acid

Load 1C, Load 2C, Load 3C, Load 4C: Cohesive Cleaning Solvent

Load 1D, Load 2D, Load 3D, Load 4D: 2% Acetonitrile (Cohesive injector Rinse solution #1)

Extraction column: 0.5 mm×5.0 cm Cyclone, Cohesive
Analytical Column: Phenomenex MAX-RP. 50 mm×2.0 mm, 4 μm

### Reporting Results/Interpreting Results

### 1. Reference Ranges:

Male: 18-64 pg/mL

Female: 18-78 pg/mL

### 2. Reportable Range:

a. High values (>1000 pg/ml) are diluted in zero standard and reassayed. The result from the assay is multiplied by the dilution factor and reported.

b. Low Values: The lowest reporting limits are 3 pg/mL for DHVD3 and 6 pg/mL for DHVD2. If both analytes are below their reporting limit the total for that sample is reported as <9 pg/mL.

### Example 2

### Representative Comparison of Results for Methods Performed with and without Affinity Purification Step

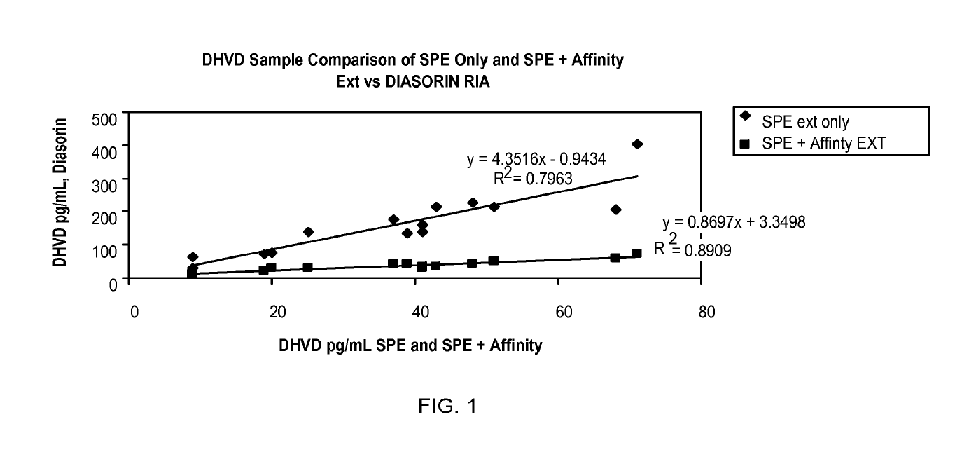

An experiment was conducted to compare the total DHVD determined in a sample using two methods: one method employed solid phase extraction purification (SPE) and derivatization steps; the second method employed solid phase extraction, affinity purification (Affinity EXT), and derivatization steps. The amount of total DHVD in the same samples was also determined using a DiaSorin™ radioimmunassay (RIA) kit. The results are shown in FIG. 1. As can be seen, the correlation coefficient as compared to RIA for the method employing the affinity purification step was significantly better than that for the method not employing such a step. In addition, the determined value of total DHVD is higher in the method that did not use affinity purification, with an approximately 4-fold higher slope than the method affinity purification. The total DHVD value may be falsely elevated due to interferences that are not removed with SPE alone.

### Example 3

### Quality Control Analysis

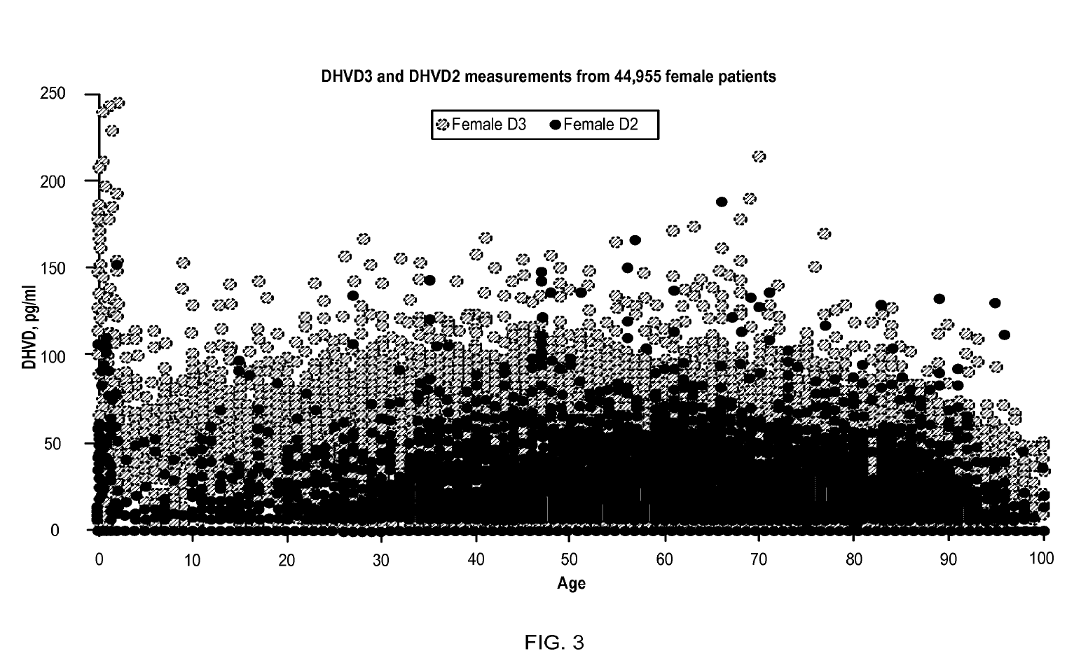

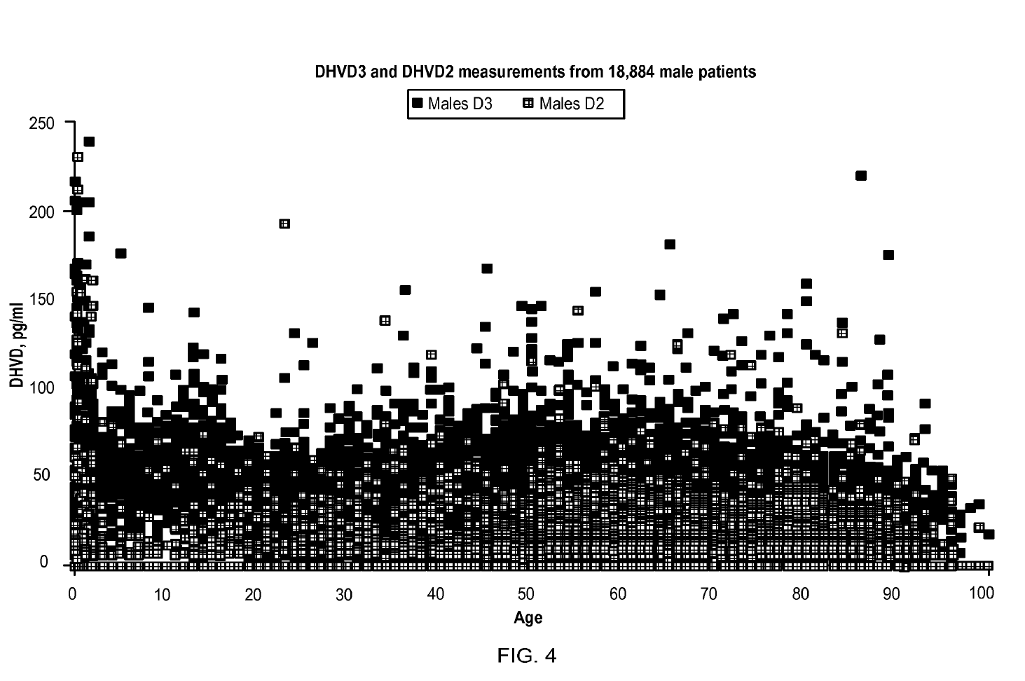

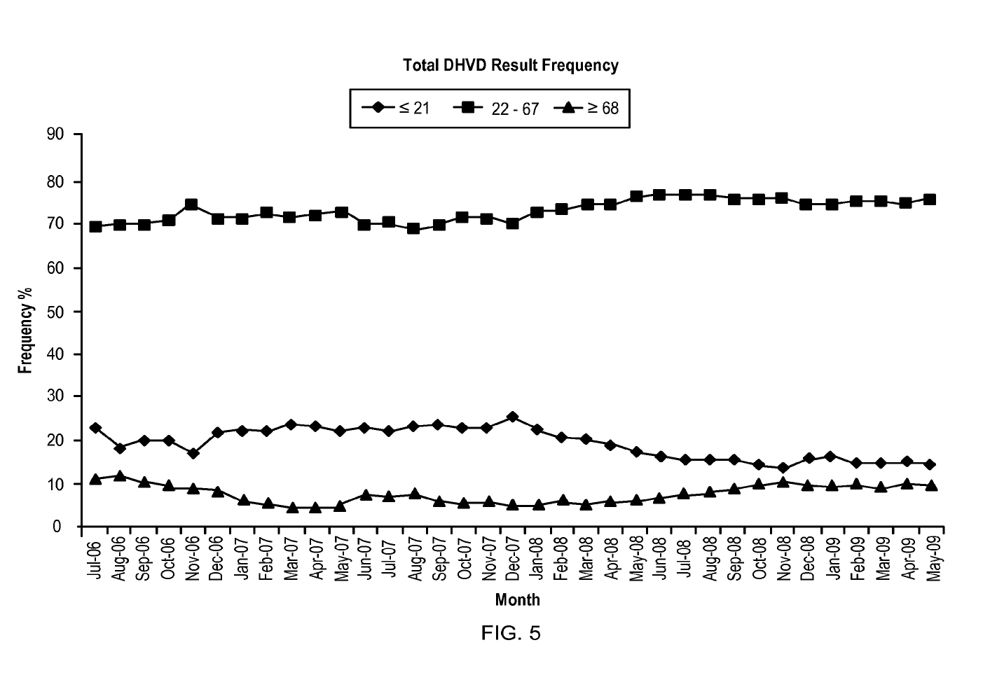

Data generated, using the method described in Example 1, from 65,000 patient samples was analyzed to demonstrate that there were no significant shifts in population levels for the analytes. Results, split by gender and plotted by age, are shown in FIGS. 3-5. Different lots of control material made from pooled serum were used to track the accuracy and stability of the assay over time. Data from these lots is listed in Table 1 and demonstrates the robustness of the assay.

TABLE 1
DHVD by LC MS/MS QC Summary

| | Estab- | Ob- | Ob- | Ob- | |
|---|---|---|---|---|---|
| | lished | served | served | served | |
| Control | mean | mean | CV | SD | Date |
| Low D2 | | | | | |
| Lot 012907 | 31 | 31.3 | 10.7 | 3.35 | Jun. 2, 2008- |
| | | | | | Jul. 2, 2008 |
| Lot 52708 | 22 | 22.7 | 11.96 | 2.72 | Jul. 3, 2008- |
| | | | | | Jun. 15, 2009 |
| Low D3 | | | | | |
| Lot 012907 | 19 | 20.6 | 10.13 | 2.09 | Jun. 2, 2008- |
| | | | | | Jul. 2, 2008 |
| Lot 52708 | 21 | 23 | 9.59 | 2.21 | Jul. 3, 2008- |
| | | | | | Jun. 15, 2009 |
| Med D2 | | | | | |
| Lot 012907 | 83 | 87.3 | 10.67 | 9.31 | Jun. 2, 2008- |
| | | | | | Jul. 2, 2008 |
| Lot 52708 | 60 | 63.6 | 10.74 | 6.83 | Jul. 3, 2008- |
| | | | | | Jun. 15, 2009 |
| Med D3 | | | | | |
| Lot 012907 | 67 | 70.3 | 10.14 | 7.12 | Jun. 2, 2008- |
| | | | | | Jul. 2, 2008 |
| Lot 52708 | 73 | 79.6 | 8.9 | 7.1 | Jul. 3, 2008- |
| | | | | | Jun. 15, 2009 |
| High D2 | | | | | |
| Lot 012907 | 287 | 298.8 | 9.09 | 27.15 | Jun. 2, 2008- |
| | | | | | Jul. 2, 2008 |
| Lot 52708 | 264 | 279.1 | 9.57 | 26.65 | Jul. 3, 2008- |
| | | | | | Jun. 15, 2009 |
| High D3 | | | | | |
| Lot 012907 | 220 | 223.7 | 10.1 | 22.59 | Jun. 2, 2008- |
| | | | | | Jul. 2, 2008 |
| Lot 52708 | 231 | 252.2 | 20.86 | 8.3 | Jul. 3, 2008- |
| | | | | | Jun. 15, 2009 |

### REFERENCES

1. Endres, D B., Rude, R. K.: Vitamin D and its Metabolites. In: Tietz Textbook of Clinical Chemistry, 3rd edition; Burtis C A, Ashwood E R (eds). W. B. Saunders Co., Philadelphia, Pa., 1999; pp. 1417-1423.
2. Bringhurst, F. R., Demay, M. B., Kronenberg, H. M.: Vitamin D (Calciferols): Metabolism of vitamin D. In: Williams Textbook of Endocrinology, 9^{th} edition; Wilson, J D, Foster D W, Kronenberg H M, Larsen P R (eds). 1998;pp. 1166-1169.

A number of embodiments of the invention have been described. Nevertheless, it will be understood that various modifications may be made without departing from the spirit and scope of the invention. Accordingly, other embodiments are within the scope of the following claims.

What is claimed is:

1. A method for determining an amount of DHVD2 in a sample, wherein the method comprises:
a) subjecting the sample to an affinity purification step comprising contacting the sample with an antibody specific for DHVD2 to form an affinity purified sample;
b) derivatizing the DHVD2 from the affinity purified sample to form a derivatized sample; and
c) subjecting the derivatized sample to a MS technique to determine the amount of DHVD2.

2. The method of claim 1, wherein the mass spectrometry technique comprises a tandem mass spectrometry (MS/MS) technique.

3. The method of claim 1, wherein the mass spectrometry technique comprises an LC-MS/MS technique.

4. The method of claim 1, wherein the derivatization comprises contacting the affinity purified sample with a Cookson-type reagent.

5. The method of claim 1, wherein the derivatization comprises contacting the affinity purified sample with a chemical selected from PTAD, MBOTAD, DMEQTAD, and MTAD.

6. The method of claim 3, wherein the LC-MS/MS technique comprises the use of a triple quadrupole instrument in Multiple Reaction Monitoring (MRM), positive-ion mode.

7. The method of claim 6, wherein the LC-MS/MS technique comprises a Q1 scan tuned to select a precursor ion that corresponds to the [M+H⁺] or [M°+H⁺] of DHVD2.

8. The method of claim 1, wherein the sample is a biological sample.

9. The method of claim 8, wherein the biological sample is a mammalian biological sample.

10. The method of claim 9, wherein the mammalian biological sample is a human biological sample.

11. The method of claim 10, wherein the human biological sample is a blood, urine, lachrymal, plasma, serum, or saliva sample.

12. The method of claim 8, wherein the sample is a food sample.

13. The method of claim 8, wherein the sample is a dietary supplement sample.

14. The method of claim 1, wherein the method further comprises precipitation of one or more proteins in the sample.

15. The method of claim 14, wherein the one or more proteins are precipitated by treating the sample with one or more reagents selected from the group consisting of acetonitrile, NaOH, and KOH.

16. The method of claim 3, wherein the LC-MS/MS technique comprises atmospheric pressure chemical ionization (APCI) or Electrospray Ionization (ESI).

17. The method of claim 1, wherein the method further comprises determining an amount of DHVD3 in the sample, wherein the antibody specific for DHVD2 is specific for DHVD3, and wherein the derivatization step derivatizes the DHVD3 in the affinity purified sample.

18. The method of claim 17, wherein the MS technique comprises a Q1 scan tuned to select, independently, precursor ions that correspond to the [M+H⁺] or [M°+H⁺] of DHVD2 and DHVD3.

19. The method of claim 18, wherein the MS technique comprises monitoring MRM precursor-product ion pair transitions having m/z values of 586.3 for DHVD2 and 574.3 for DHVD3.

20. The method of claim 17, wherein the method comprises determining the amounts of DHVD2 and DHVD3 using a standard calibration curve.

21. The method of claim 17, further comprising the use of DHVD2-d6 and DHVD3-d3 as internal standards.

22. The method of claim 21, wherein the DHVD2-d6 internal standard has a MRM parent-daughter ion pair transition m/z values of 592.3/314.2 and the DHVD3-d3 internal standard has a MRM parent-daughter ion pair transition m/z values of 577.3/317.2.

23. A method for determining whether or not a mammal has a vitamin D deficiency, the method comprising determining the amount of DHVD2 and DHVD3 in a sample from the mammal.

24. A method for determining whether or not a mammal has hypervitaminosis D, the method comprising determining the amount of DHVD2 and DHVD3 in a sample from the mammal.

25. A method for monitoring vitamin D replacement therapy in a mammal, the method comprising determining the amount of DHVD2 in a sample from the mammal.
